(19) United States
(12) Patent Application Publication
(10) Pub. No.: US 2012/0083422 A1
(43) Pub. Date: Apr. 5, 2012

(54) Glioblastoma Biomarkers, and Methods and Compositions

(76) Inventors: Claudius Mueller, Gainesville, VA (US); Lance Liotta, Bethesda, MD (US); Emanuel Petricoin, Gainesville, VA (US); Virginia Espina, Rockville, MD (US)

(21) Appl. No.: 13/152,204

(22) Filed: Jun. 2, 2011

Related U.S. Application Data
(60) Provisional application No. 61/350,605, filed on Jun. 2, 2010.

(51) Int. Cl. C12Q 1/02; C40B 30/04; C40B 30/06

(52) U.S. Cl. 506/9; 435/29; 506/10

(57) ABSTRACT

Glioblastoma multiforme biomarkers, methods, and compositions are provided. Methods of selecting a treatment for a patient with a brain neoplasm, including Glioblastoma multiforme, are provided. Methods of treating a patient at risk for Glioblastoma multiforme are provided.

### CROSS-REFERENCE TO RELATED APPLICATIONS

This application claims the benefit of U.S. Provisional Application No. 61/350,605, filed Jun. 2, 2010, entitled “Biomarkers for Glioblastoma and Treatment,” which is hereby incorporated by reference in its entirety.

### BRIEF DESCRIPTION OF THE SEVERAL VIEWS OF THE DRAWINGS

FIG. 1 is a diagram showing the stratification of a patient population based on survival time.

FIG. 2 (A-G) are plots of up- or downregulated protein endpoints based on protein ratios and relative intensity in the Discovery set of samples (short term, n=15 and long term, n=11) as found by mean comparison analysis. All endpoint markers (A-G) showed a significant (p<0.05) difference between the short and long term groups in the Discovery set, where 2E showed the most significant difference (p<0.01).

FIG. 3 (A-H) are plots of up- or downregulated protein endpoints in the Discovery set of samples as indicated by Kaplan-Meier analysis. All endpoint markers (A-H) showed a significant (p<0.05) difference, where 3C and 3G showed the most significant difference (p<0.01).

FIG. 4 (A,B) are graphical representations showing significantly altered protein ratios in the Discovery set of samples, as found by Kaplan-Meier analysis (A) or mean comparison (B) between Numb versus PAK1 Thr432/PAK2 Thr402. Correlation plots (C, D) demonstrate significant protein correlations between Numb versus PAK1 Thr432/PAK2 Thr402 within short-term survivors (C) but is lost in long-term survivors (D).

FIG. 5 (A,B) are graphical representations showing significantly altered protein ratios in the Discovery set of samples, as found by Kaplan-Meier analysis (A) or mean comparison (B) between PKC alpha Ser657 versus Adducin Ser662. Correlation plots (C, D) demonstrate significant protein correlations between PKC alpha Ser657 versus Adducin Ser662 within short-term survivors (C) but is lost in long-term survivors (D).

FIG. 6 (A,B) are graphical representations that did not show significantly altered protein ratios in the Discovery set of samples, as found by Kaplan-Meier analysis (A) or mean comparison (B) between Numb versus Src Tyr527. Correlation plots (C,D) demonstrate significant protein correlations between Numb versus Src Tyr527 within long-term survivors (D) but is lost in short-term survivors (C).

FIG. 7 (A,B) are graphical representations showing significantly altered protein ratios in the Discovery set of samples, as found by Kaplan-Meier analysis (A) or mean comparison (B) between Bax versus cAbl Thr735. Correlation plots (C,D) demonstrate significant protein correlations between Bax versus cAbl Thr735 within short-term survivors (C) but is lost in long-term survivors (D).

FIG. 8 is a flow diagram of the steps for generating a rank score (“CFLN score”) which was calculated from four proteins: cPLA2 Ser505, FAK Tyr576/577, LC3B and Numb. Using the discovery set a cutoff of 1.390244was calculated, which separated limited from extended survivors with a sensitivity of 80% and specificity of 100%. Application of this cutoff to the validation set resulted in a sensitivity of 100% and 86%.

FIG. 9 is an image of the raw data from the RPMA analysis showing five (1-5) subgroups that are identified with unique signaling activation signatures that are drug targets for therapy.

### DESCRIPTION OF EMBODIMENTS

In one aspect, methods are provided for assessing whether a potential therapeutic agent may be useful for the treatment of preneoplastic lesions of the brain comprising administering in vitro the potential therapeutic agent to the population of brain cells as described herein, culturing the cells, and determining whether the therapeutic agent inhibits the growth of the cells, proliferation of the cells or tendency of the cells to invade or metastasize. In one embodiment, the determination step involves evaluating exposed brain cells for the phosphorylation state of two or more endpoints in a signal pathway having the target of one or more candidate therapeutics.

In another aspect of an embodiment, a method of selecting a treatment for a patient with a brain neoplasm is provided comprising incubating a brain tissue sample from said patient with one or more candidate therapeutics as described herein. In a further embodiment, at least one patient with the brain neoplasm is a patient with Glioblastoma multiforme. In an additional teaching, determining the activation status of an at least one protein may include evaluating the phosphorylation state of two or more endpoints in a signal pathway comprising the target of said one or more candidate therapeutics.

According to embodiments, determining activation status of the at least one protein may include evaluating the phosphorylation state of two or more endpoints in a signal pathway influencing angiogenesis, cytoskeleton and cytoskeletal reorganization, apoptosis, growth factor signaling, and cytoplasmic kinases.

According to embodiments, short and long-term survivors may be determined.

In an additional embodiment, activated proteins may include, VEGFR2, eNOS, HIF1 alpha, adducin, beta actin, PAK1/2, BCL-2, caspase 3, cKIT, IGF, IRS, AKT, PKC alpha, FAK, and PLK1.

In an additional embodiment, the activation status may be determined by molecular analysis of a putative target of the selected therapeutic. In a further embodiment, the determination by molecular analysis may include determining by reverse phase microarray (RPMA), suspension bead array, ELISA, flow cytometry, immunoassay and high resolution mass spectroscopy.

In one aspect, a method of identifying potential treatments for patients with brain tumors may include incubating a brain tissue sample from said patient with one or more candidate therapeutics, stopping the incubation and applying a fixative to the stopped incubation, determining the activation status of the at least one protein, and/or using the determination to select a treatment that advantageously impacts brain tissue.

In another aspect, a patient may be a patient with Glioblastoma multiforme.

Additionally, in another aspect of an embodiment, a method of treating a patient at risk for Glioblastoma multiforme may include selecting a treatment as set forth hereinabove and administering said treatment to a patient in need thereof.

In an aspect of an embodiment, treatment may include administering a therapeutically effective amount of at least one anti-EGFR family therapy combined with at least one AKT-MTOR inhibitor, at least one RET inhibitor, and at least one SMAD inhibitor.

In an additional aspect, a method of treating a patient at risk for Glioblastoma multiforme may include administering to said patient a JAK-STAT inhibitor.

In a further embodiment, administering to a patient includes administering a compound and a therapeutically effective amount.

In an additional aspect, a method of treating a patient at risk for Glioblastoma multiforme may include administering a therapeutically effective amount of at least one of at least one Cyclin B, D inhibitor combined with at least one AKT inhibitor, at least one ERK inhibitor, at least one IGFR inhibitor, at least one apoptosis activator, and at least one GLEEVEC-type inhibitor.

In an additional aspect, a method of treating a patient at risk for Glioblastoma multiforme may include administering a therapeutically effective amount of at least one GLEEVEC-type inhibitor includes at least one of at least one EGFR inhibitor, at least one CKIT inhibitor, and at least one CABL inhibitor.

In an additional teaching, a method of treating a patient at risk for Glioblastoma multiforme may include administering a therapeutically effective amount of at least one of an at least one BAK inhibitor, and at least one BCL2 inhibitor.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle the invention and cannot be expected to specifically illustrate the application of this invention to all the examples were be obviously useful to those skilled in the prior art.

### EXAMPLES

### Example 1

### Determination of Discovery and Validation Sample Sets

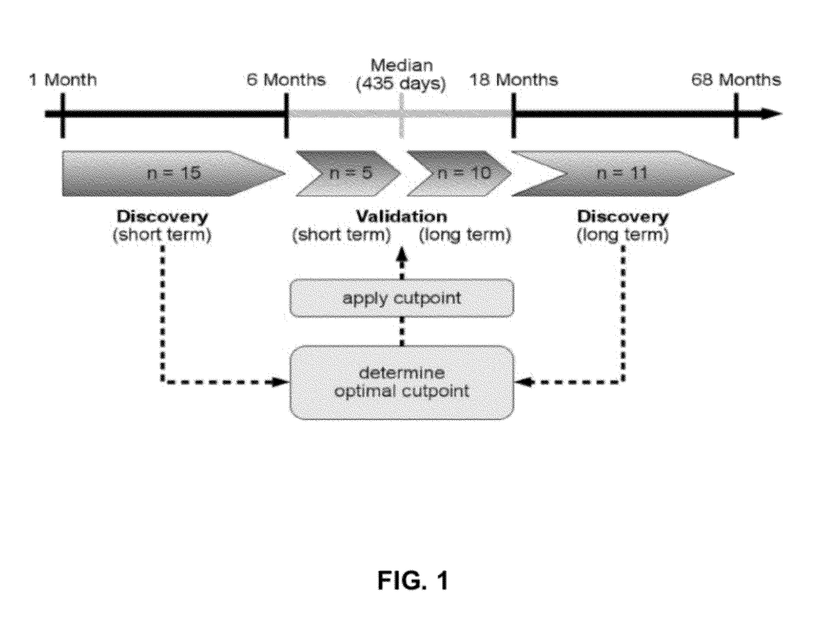

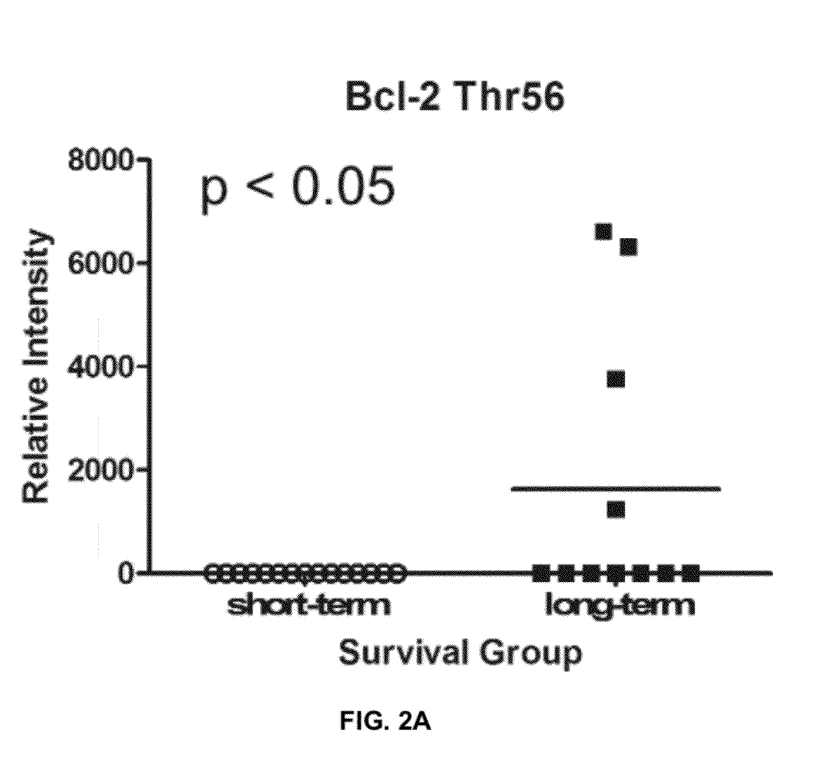

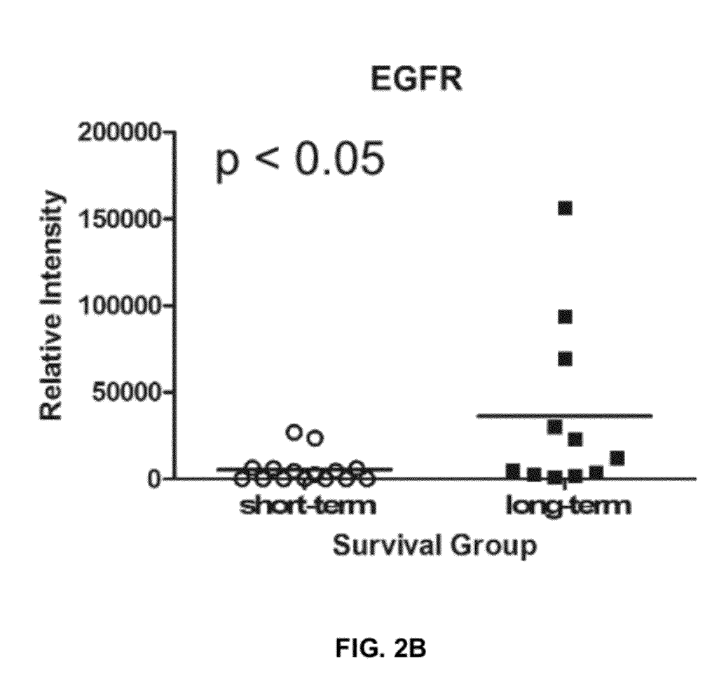

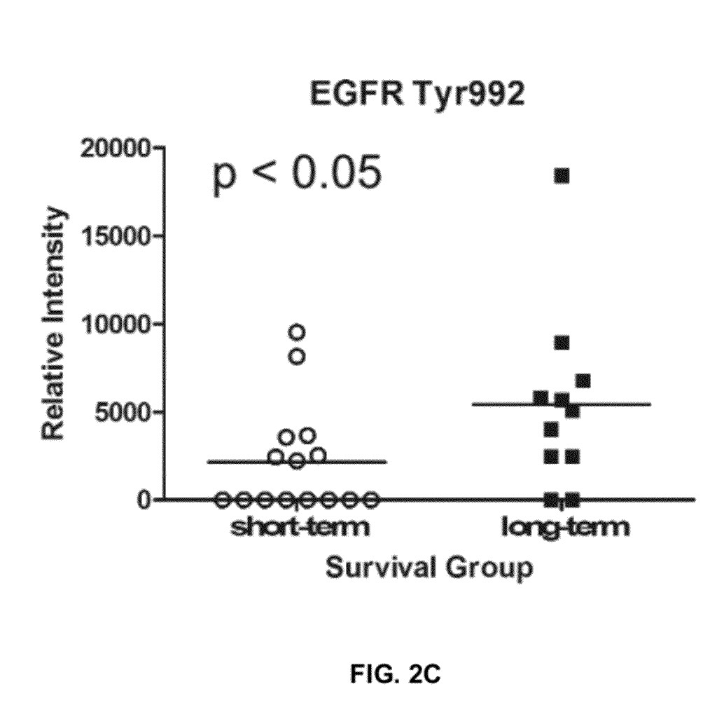

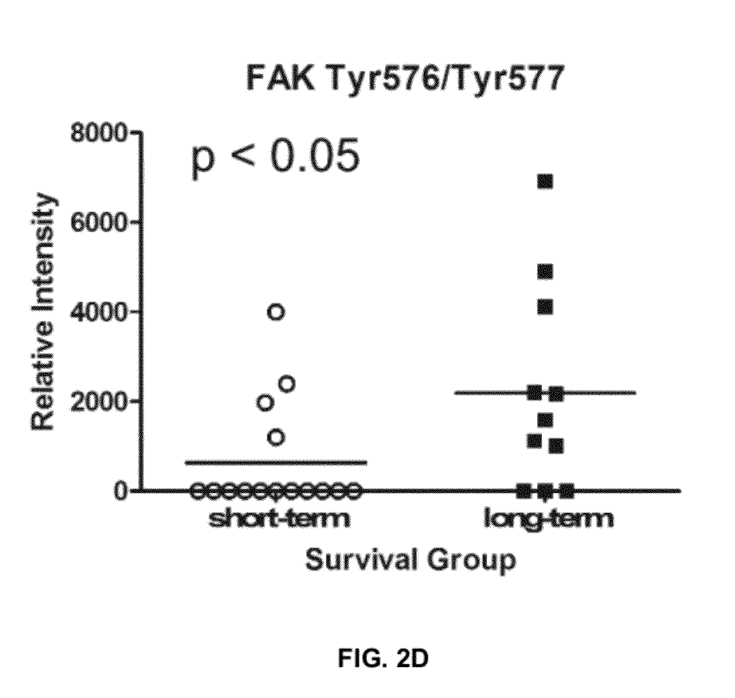

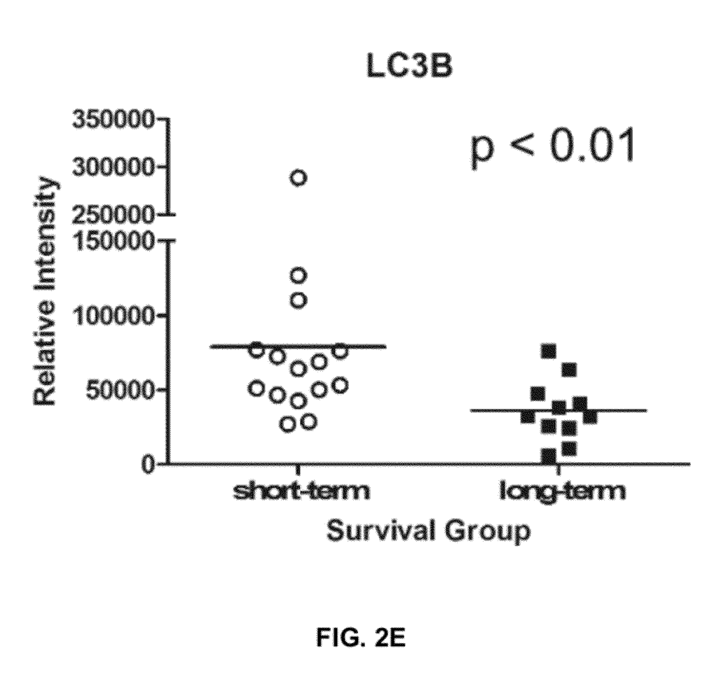

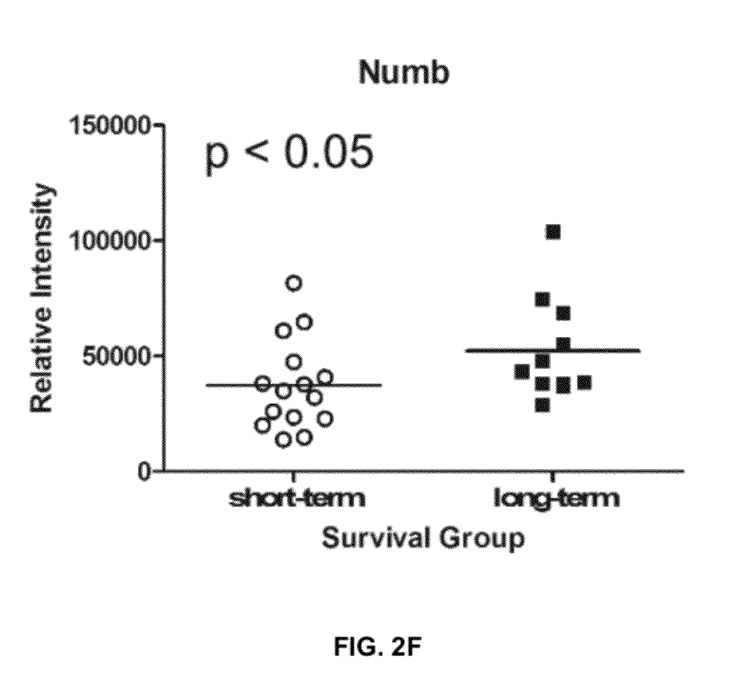

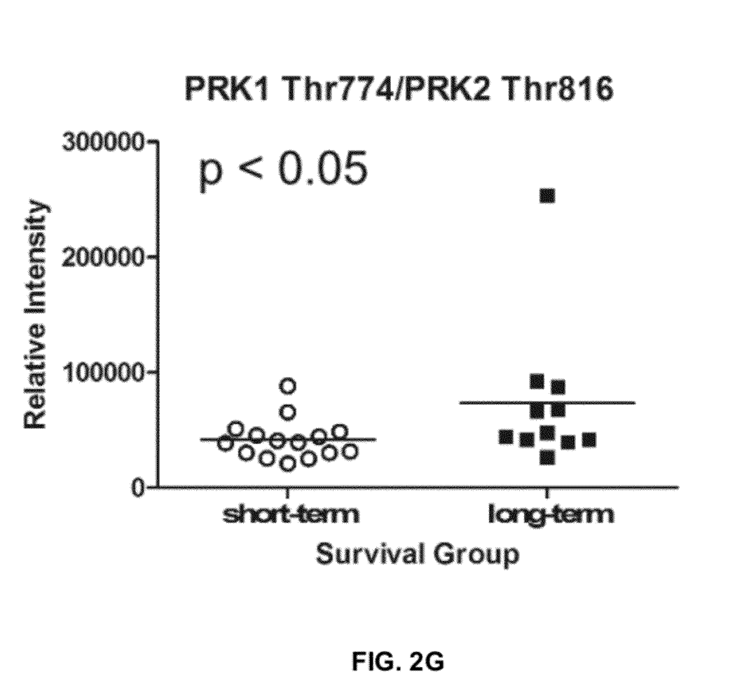

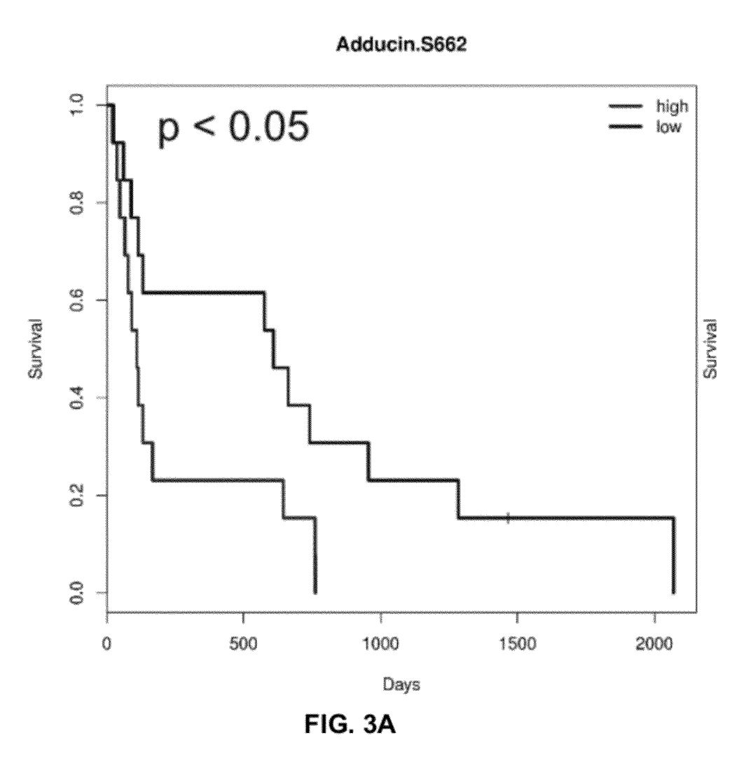

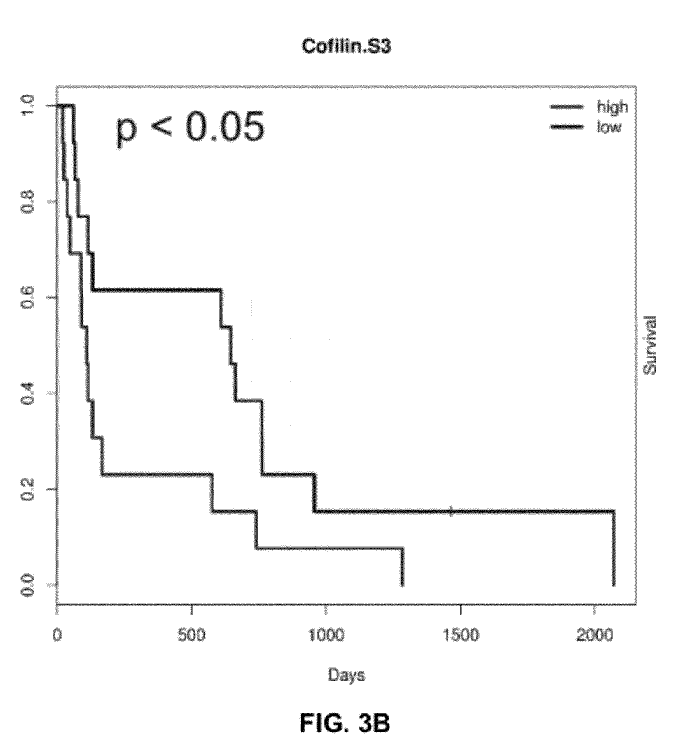

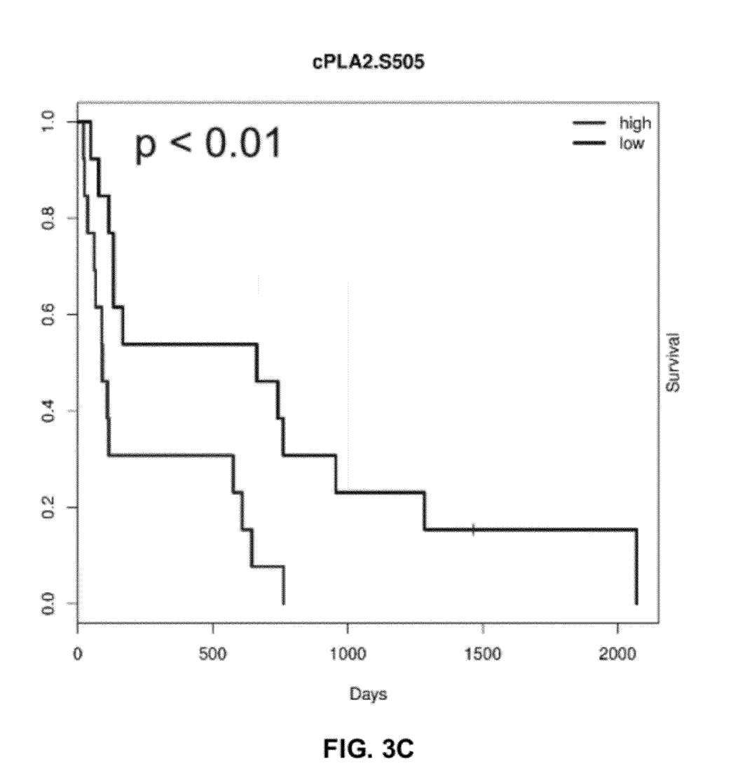

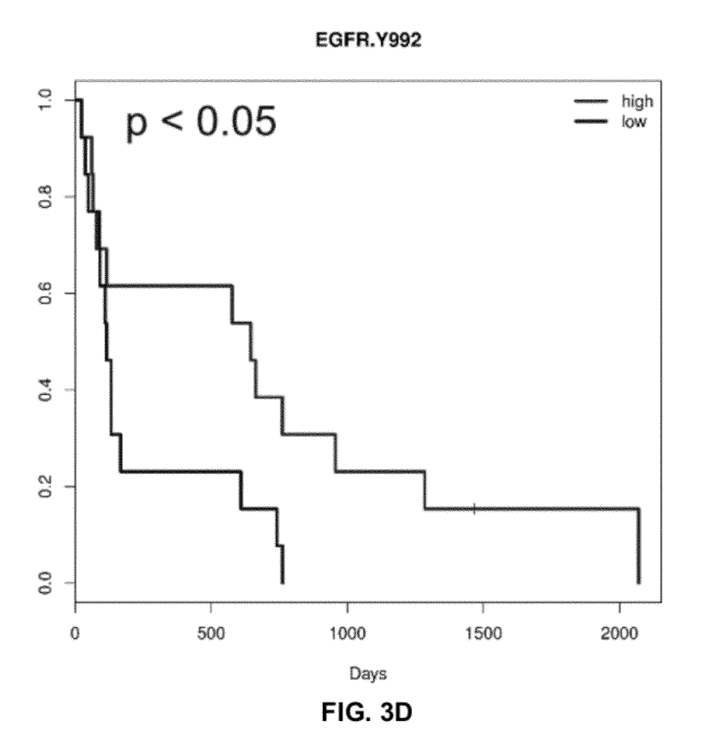

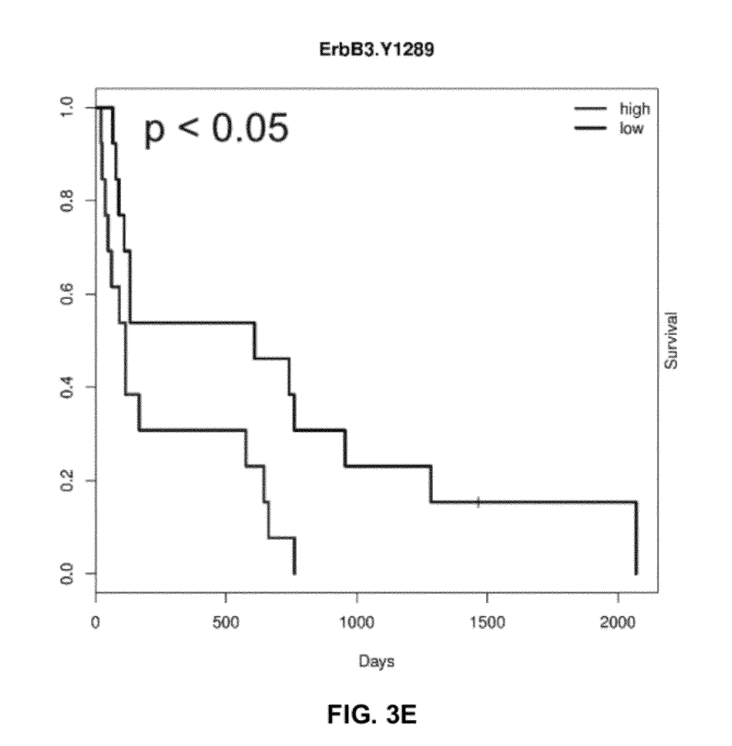

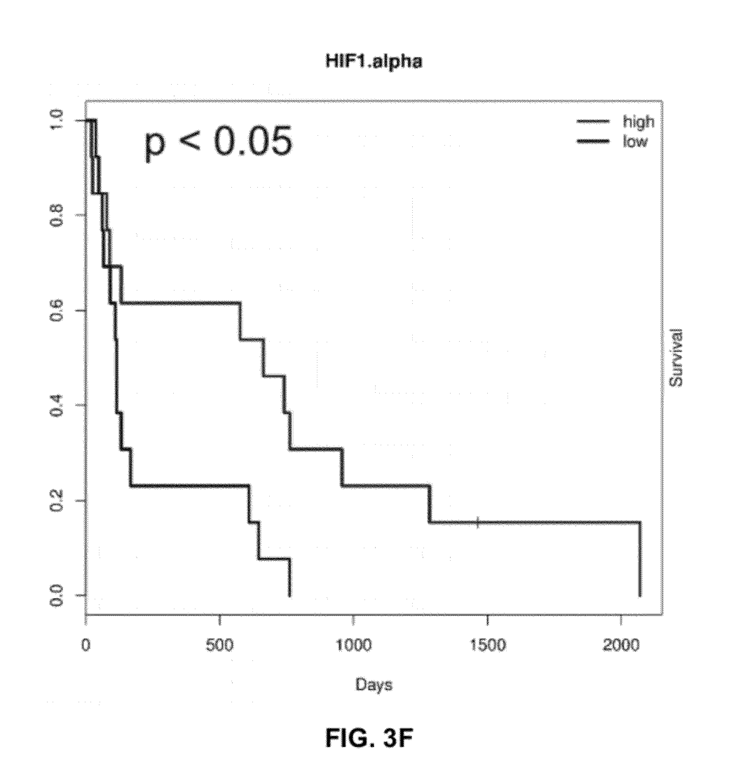

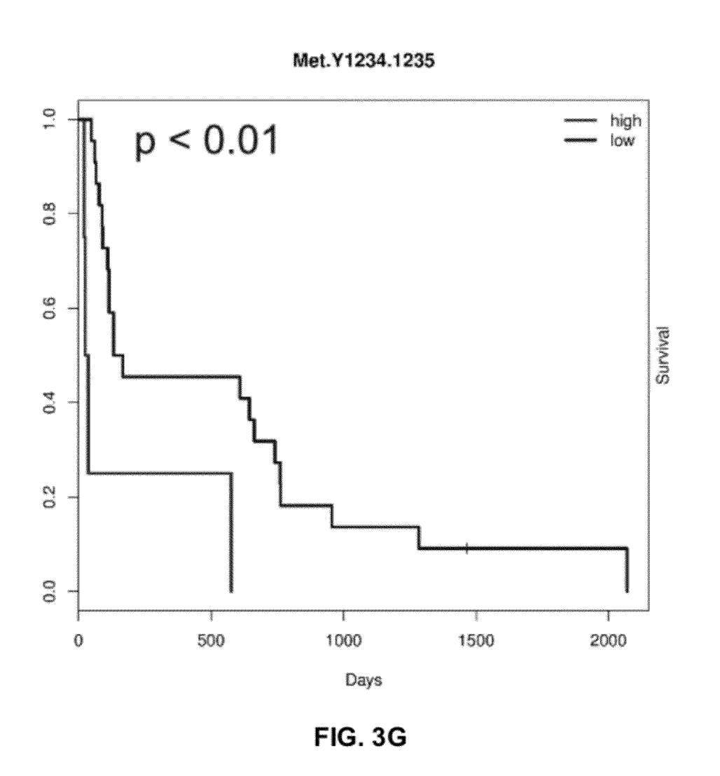

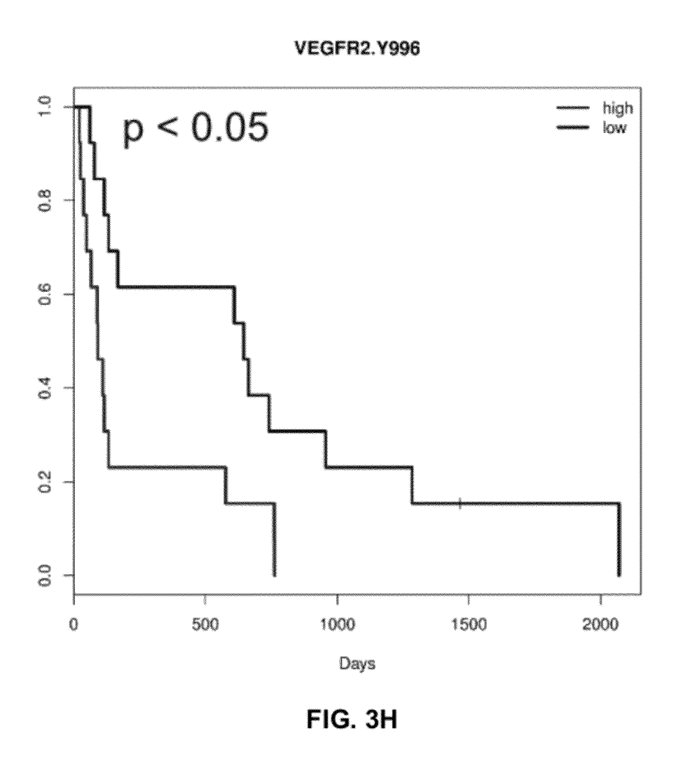

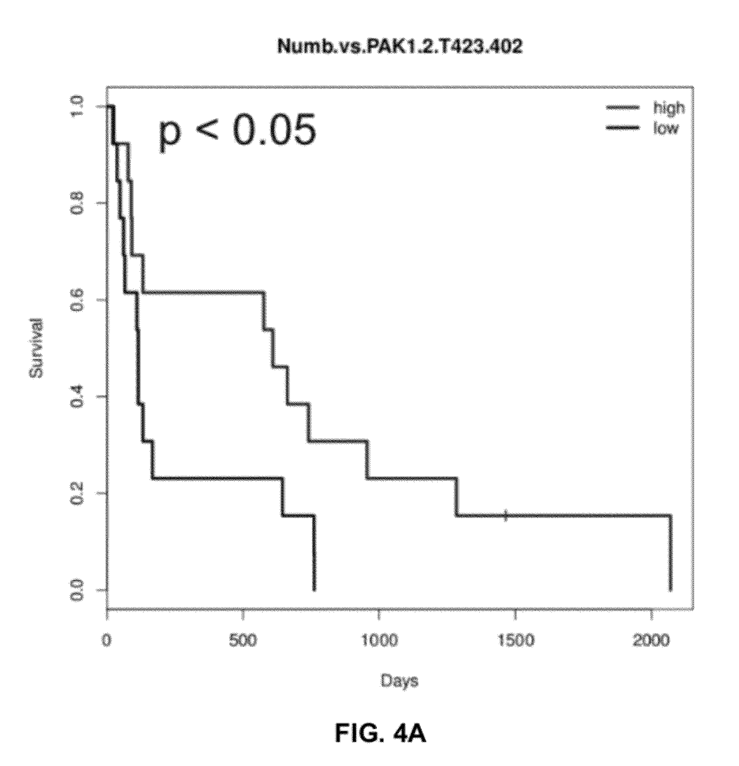

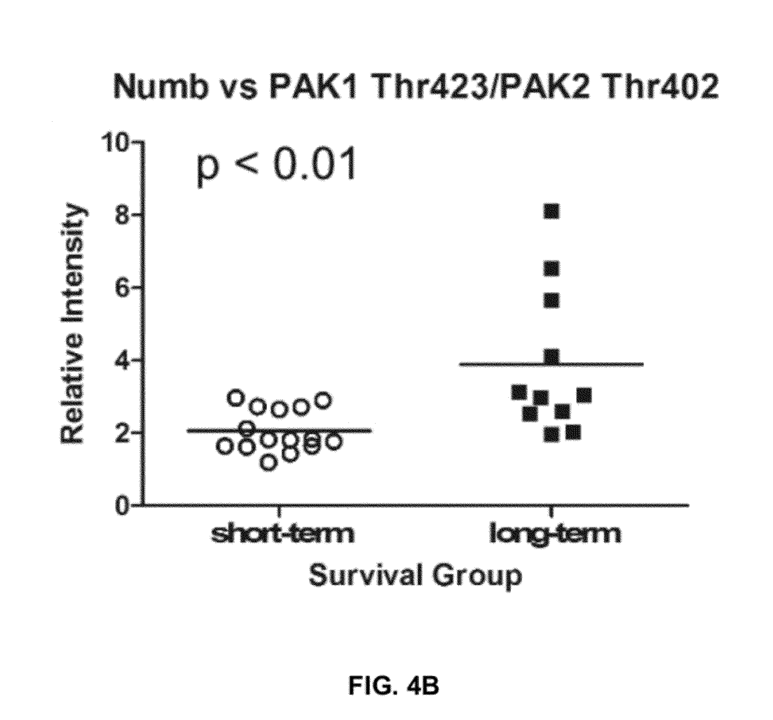

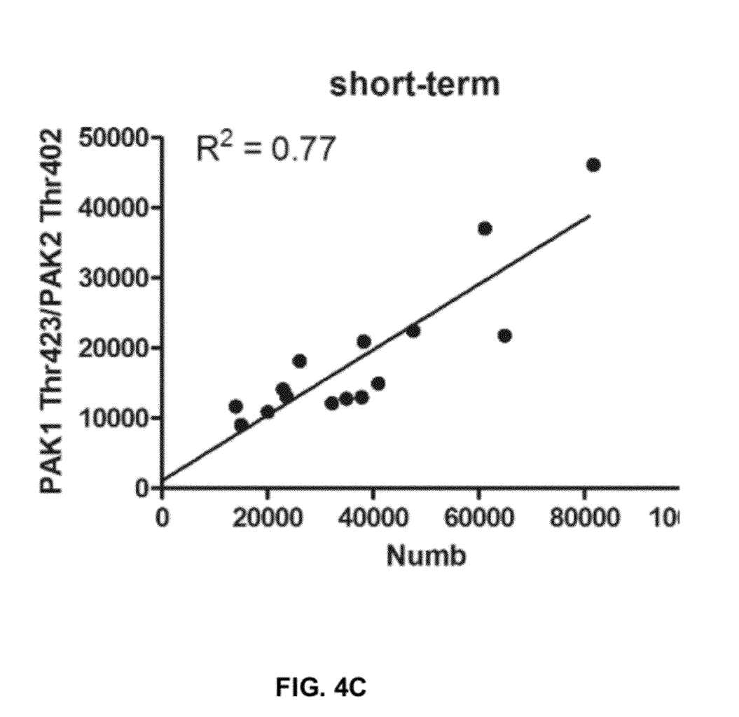

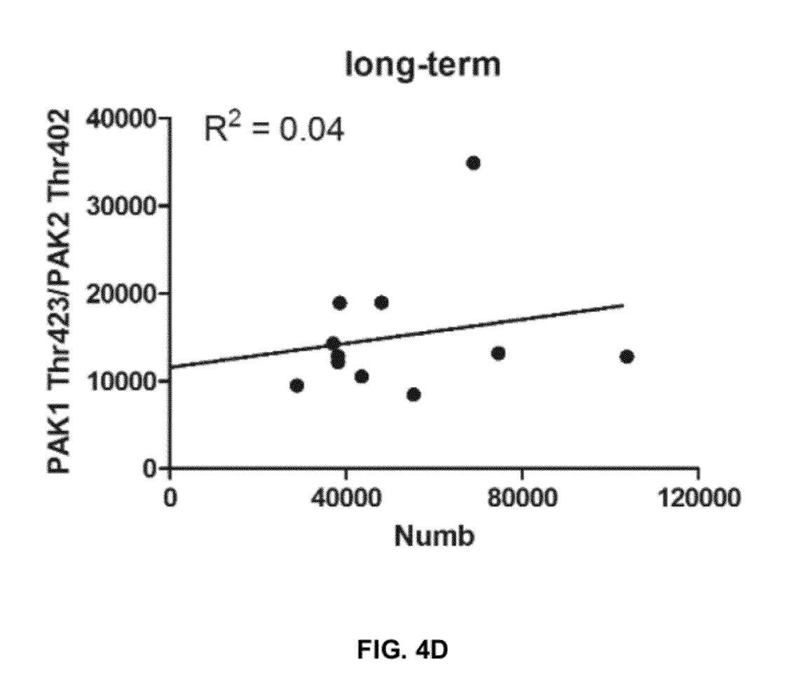

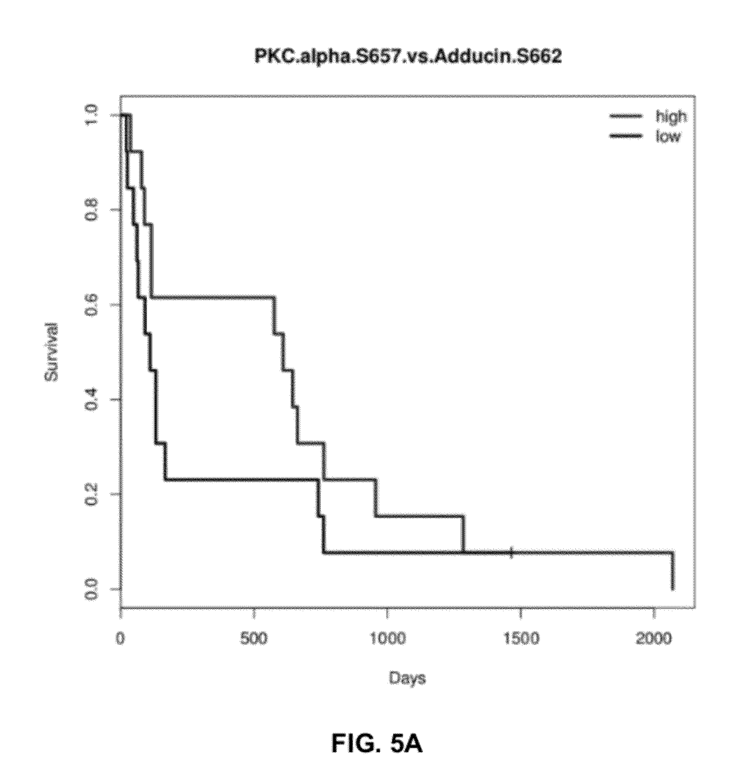

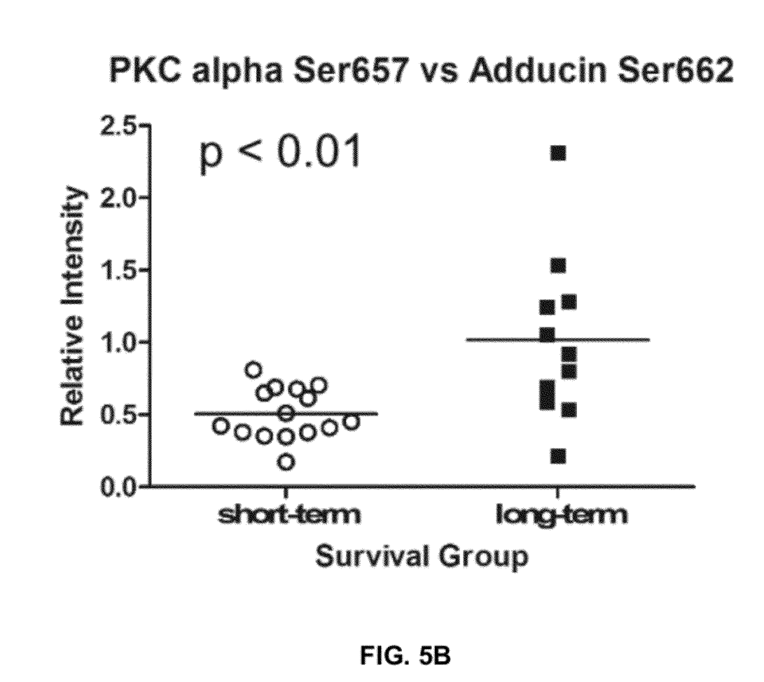

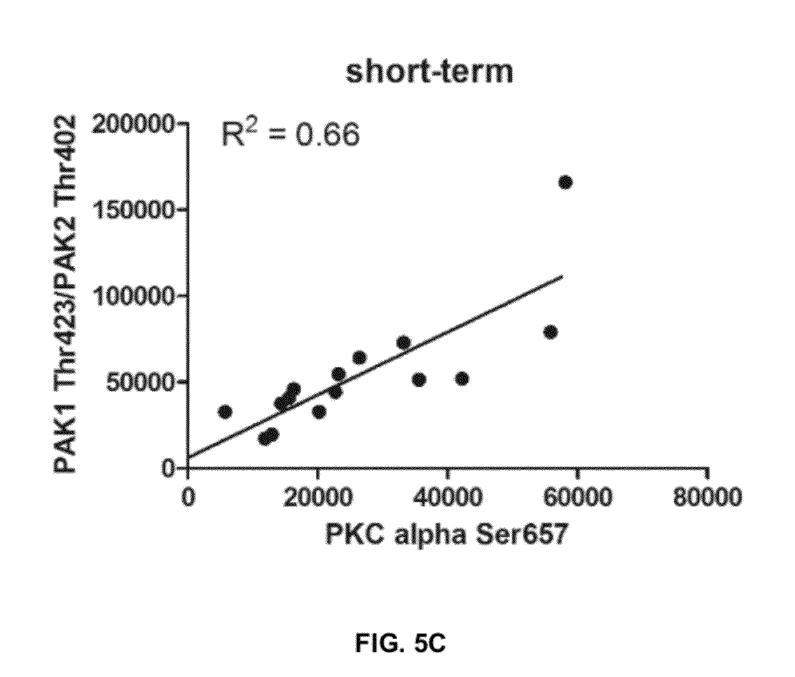

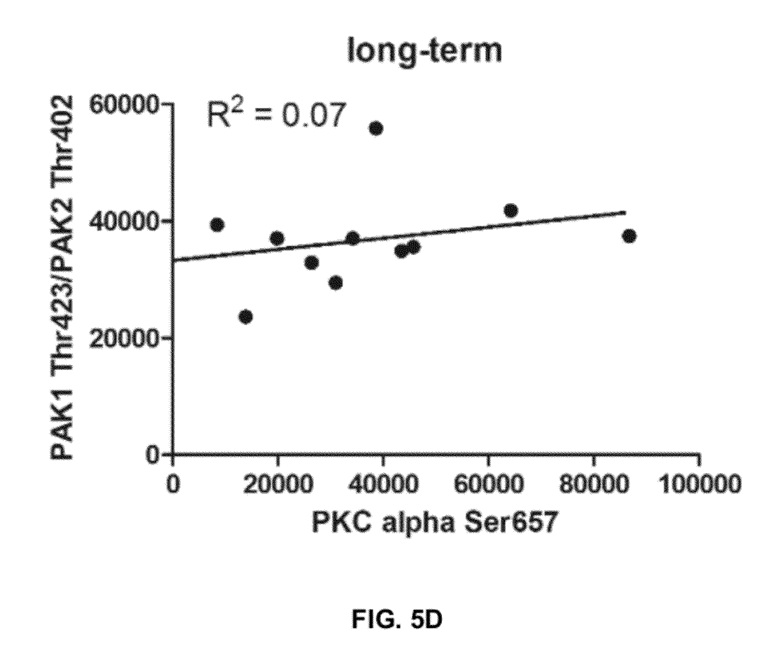

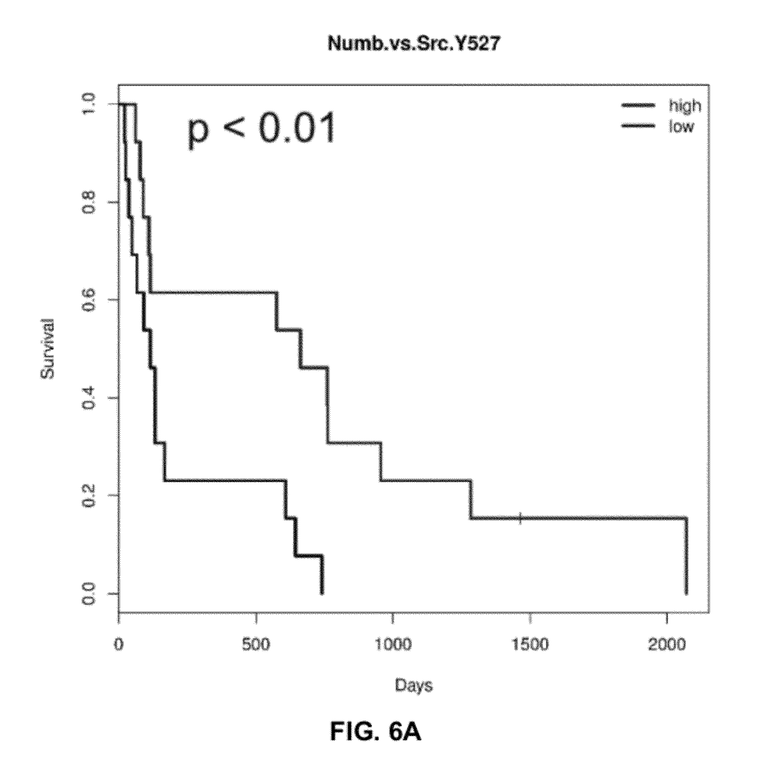

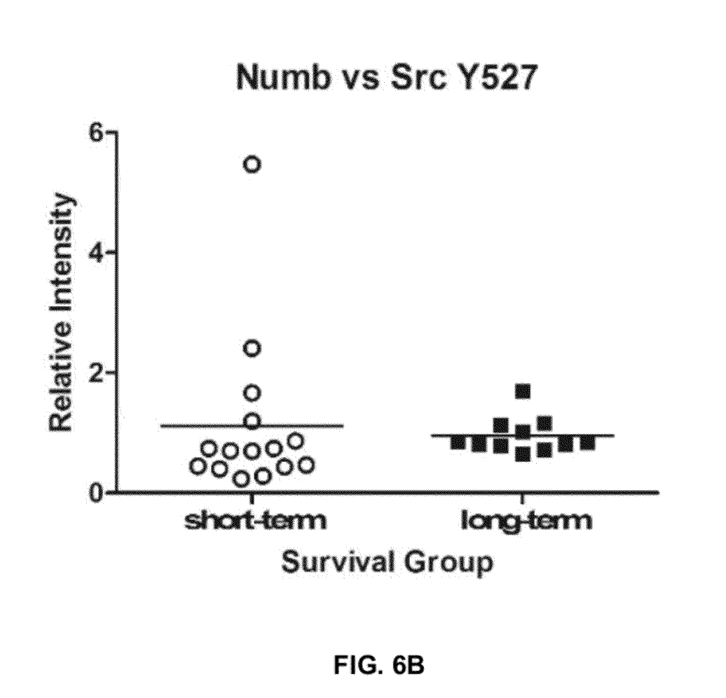

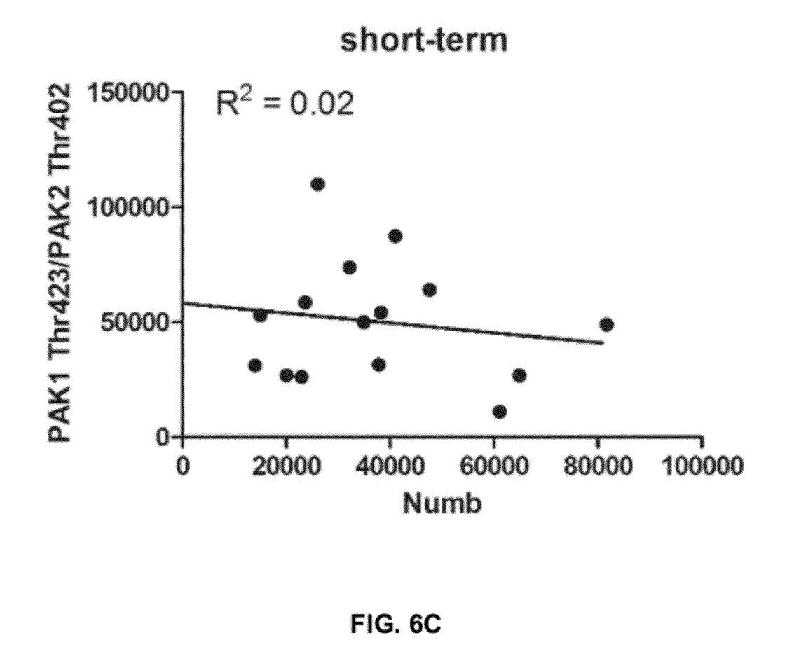

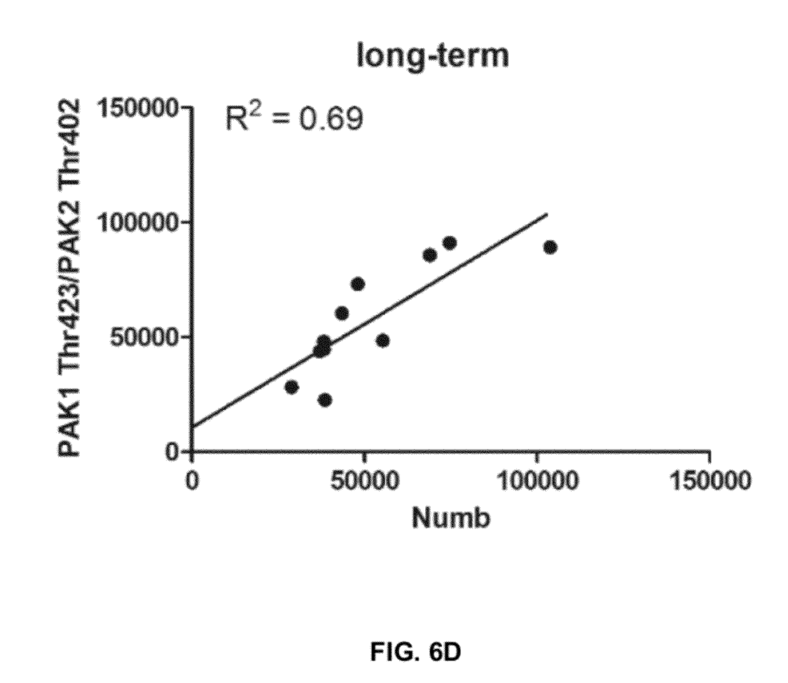

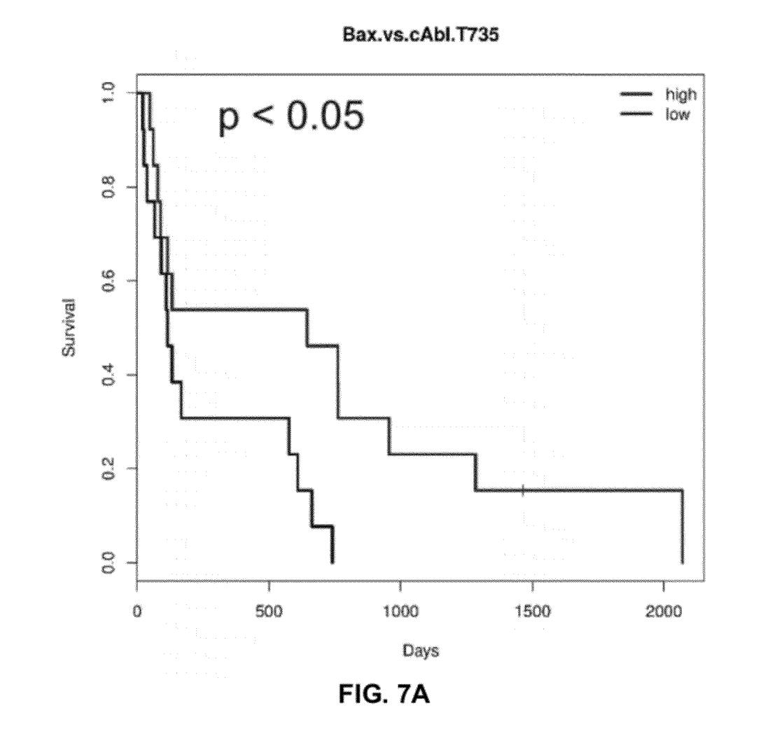

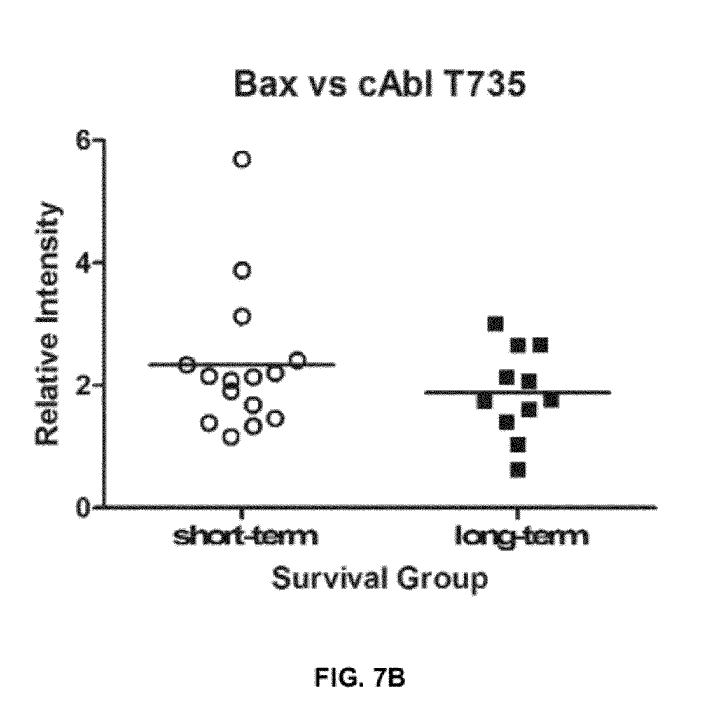

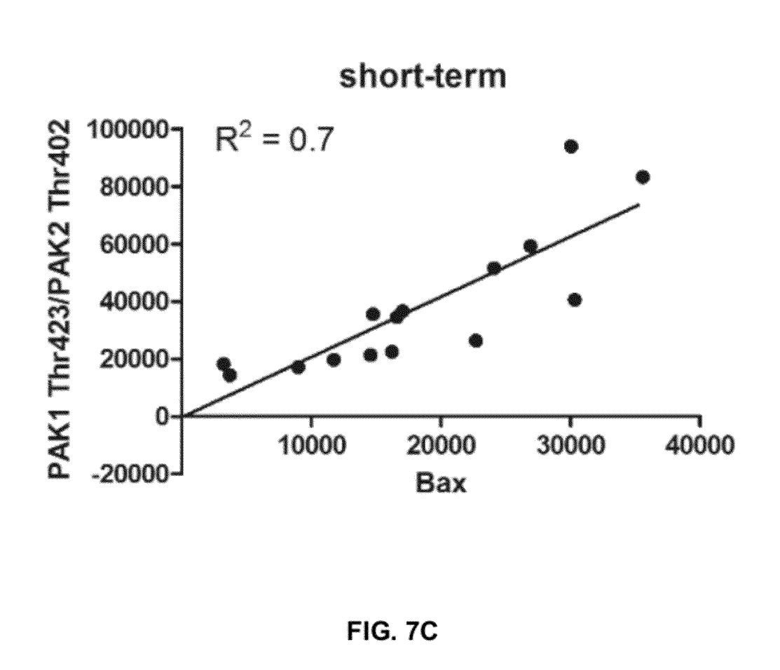

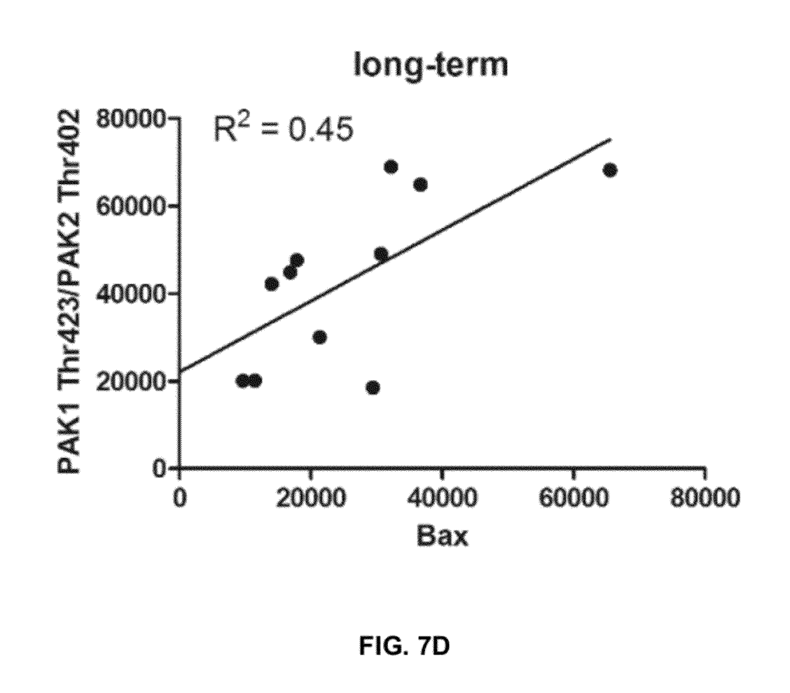

Patient samples were separated into a discovery set (n=26) and a validation set (n=15). Inclusion criteria for the discovery set were a patient survival time of either shorter than 6 months (n=15) or longer than 18 months (n=11). These criteria were based on the objective to separate limited from extended survivors with 12 months of overlap-free time. Samples with a patient survival time of 6 to 18 months (n=15) were combined to make up the validation set. Separation into limited and extended survivors within the validation set was achieved by applying the median survival time (434 days) of the validation set of patients. Thus, a cleanly separated sample set of limited and extended survivors was used to discover differences in protein abundance and phosphorylation, and the indicated differences were then applied to a sample set with much smaller differences between survival times to more closely match the general population average. (See FIG. 1).

### Example 2

### Laser Capture Microdissection

Frozen sections (8 μm) were prepared on glass microscope slides (Thermo Fisher, Atlanta, Ga.) and stored at −80° C. prior to staining and microdissection. Prior to laser capture microdissection (LCM) the frozen section slides were fixed briefly in 70% ethanol, rinsed in water, stained with Mayer's hematoxylin (Sigma-Aldrich), developed in Scott's Tap Water (Thermo Fisher), and dehydrated in an ethanol gradient (70, 95, and 100%) with a final rinse in xylene (Sigma). After a brief period of air drying non-necrotic tumor cell populations were microdissected with an Arcturus XT LCM (Molecular Devices). Microdissected cells were stored at ∥80° C. until lysis with an extraction buffer consisting of a 10% (v/v) solution of Tris (2-carboxyethyl) phosphine (TCEP; Pierce, Rockford, Ill.) in Tissue Protein Extraction Reagent (T-PER™, Pierce)/2X SDS Tris-glycine buffer (Invitrogen, Carlsbad, Calif.). Cell lysates were incubated at 100° C. for 8 minutes and frozen at −80° C. until further analysis.

### Example 3

### Reverse Phase Protein Microarrays

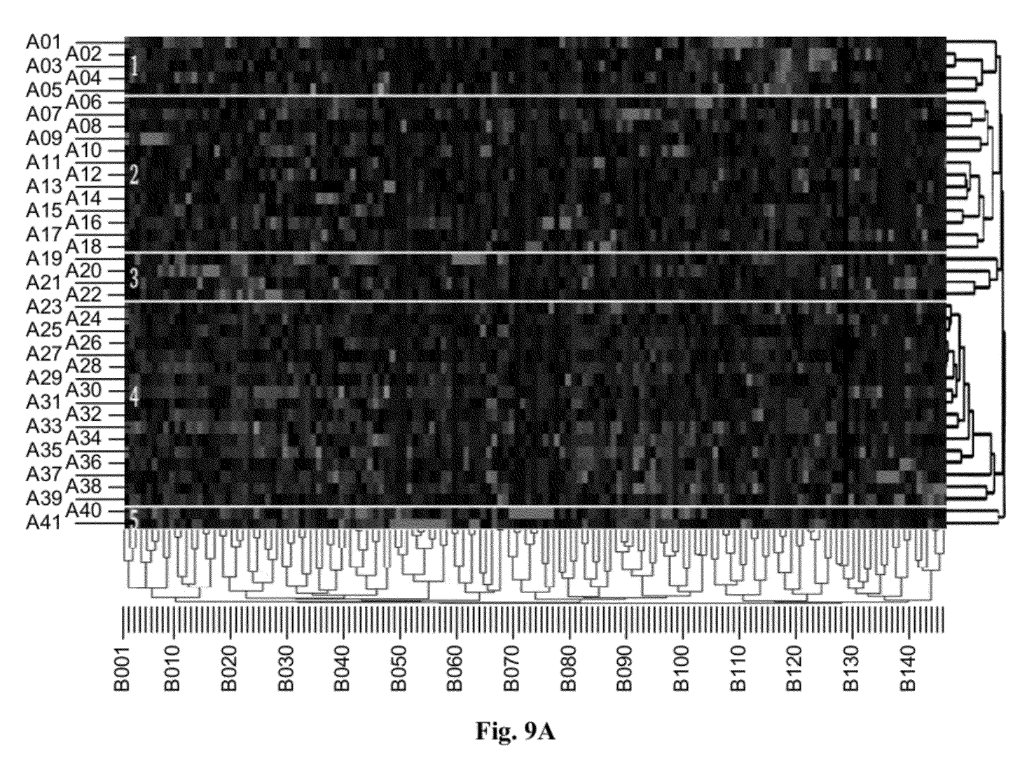

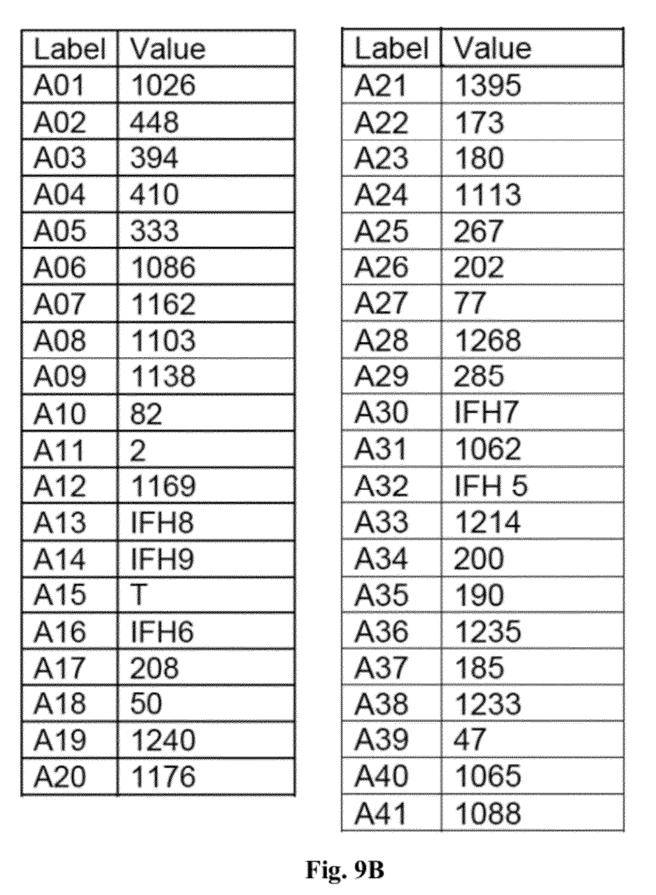

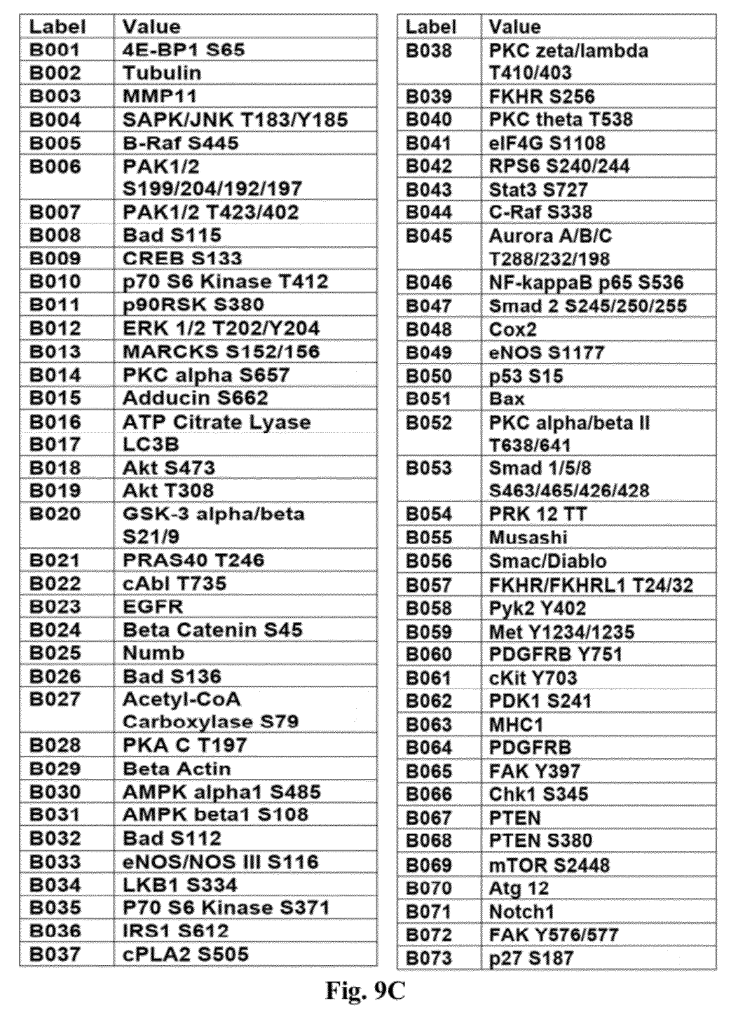

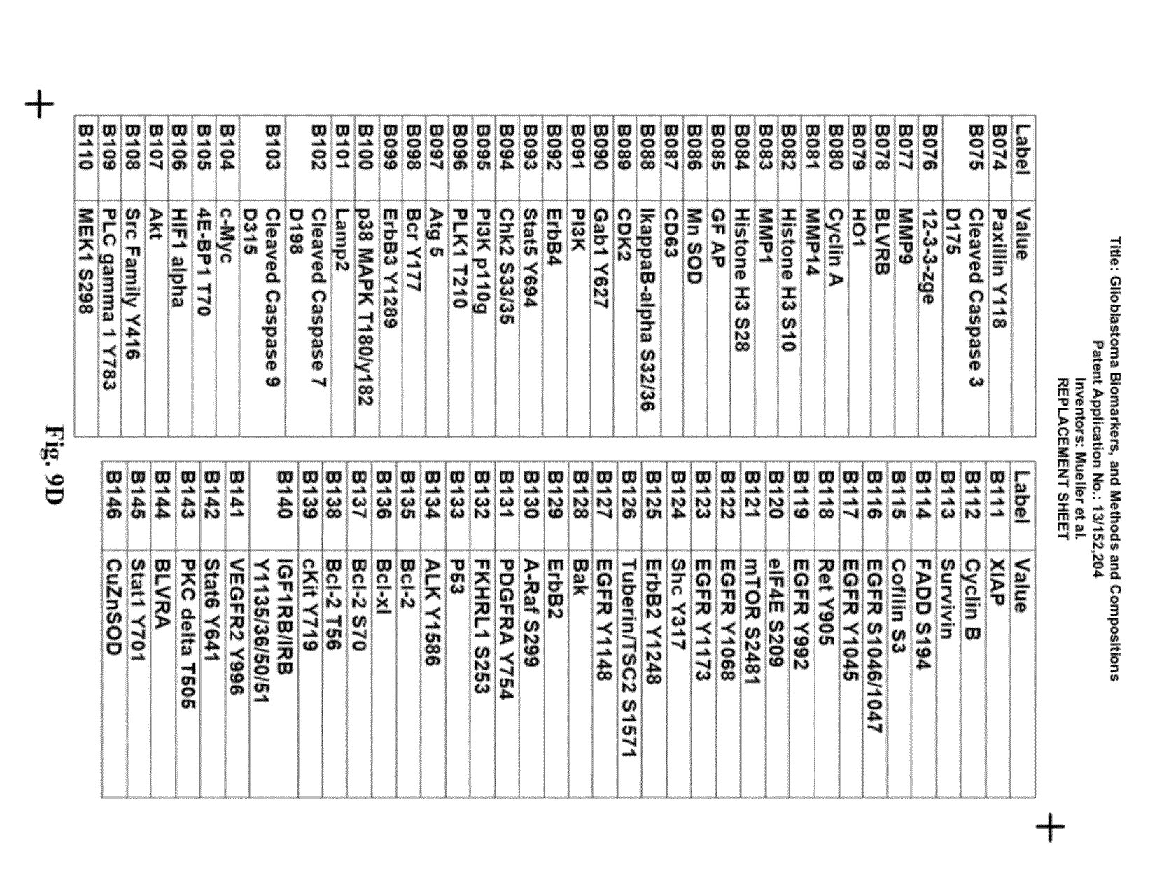

Microdissected tissue lysates were printed in triplicate on glass backed nitrocellulose array slides (Schott, Lexington, Ky.) using an Aushon 2470 arrayer (Aushon BioSystems, Burlington, Mass.) equipped with 185 μm pins. Serial two-fold dilutions of bovine serum albumin (concentration 1.0 mg/mL) were printed on the arrays as a total protein standard. Cellular lysates prepared from HeLa+Pervanadate (Becton Dickinson, Franklin Lakes, N.J.), Jurkat+Calyculin, or Jurkat+Etoposide (Cell Signaling Technology, Danvers, Mass.) cell lines were printed on each array for quality control assessments. The slides were stored with desiccant (Drierite, W. A. Hammond, Xenia, Ohio) at −20° C. prior to immunostaining. (See FIG. 9).

### Example 4

### Reverse Phase Protein Microarray Immunostaining

RPMA staining was performed on a Dako Autostainer per manufacturer's instructions (CSA kit, Dako). Each slide was incubated with a single primary antibody at room temperature for 30 minutes. Antibodies were validated by western blotting as previously described. The negative control slide was incubated with antibody diluent. Secondary antibody was goat anti-rabbit IgG H+L (Vector Labs, Burlingame, Calif.) or rabbit anti-mouse IgG (Dako). Total protein per microarray spot was determined with Sypro Ruby protein blot stain (Invitrogen/Molecular Probes) per manufacturer's directions and imaged with a CCD camera (Nova Ray, Alpha Innotech, San Leandro, Calif.). (See FIG. 9).

### Example 5

### Statistical Analysis

Statistical significance was determined using Student's t test (if the data showed normal distribution) or Wilcoxon's Rank Sum (for non-normal distributed data sets). Kaplan-Meier analysis was performed based on the median and the log-rank test was used to determine p-values of the survival analysis. A p<0.05 was considered to indicate statistical significance. The significance of endpoint correlation was calculated using Spearman's Rho analysis on the discovery set of samples. Protein ratios were filtered for having a Spearman's Rho coefficient>0.85 and p<0.05. Mean comparisons and Kaplan-Meier analyses were then performed on the retained protein ratios using both ratio arrangements (a/b and b/a). If p<0.05 for both ratio arrangements for either the mean comparison or the Kaplan-Meier/log-rank analysis, protein ratios were considered to be significantly different between limited and extended survivors.

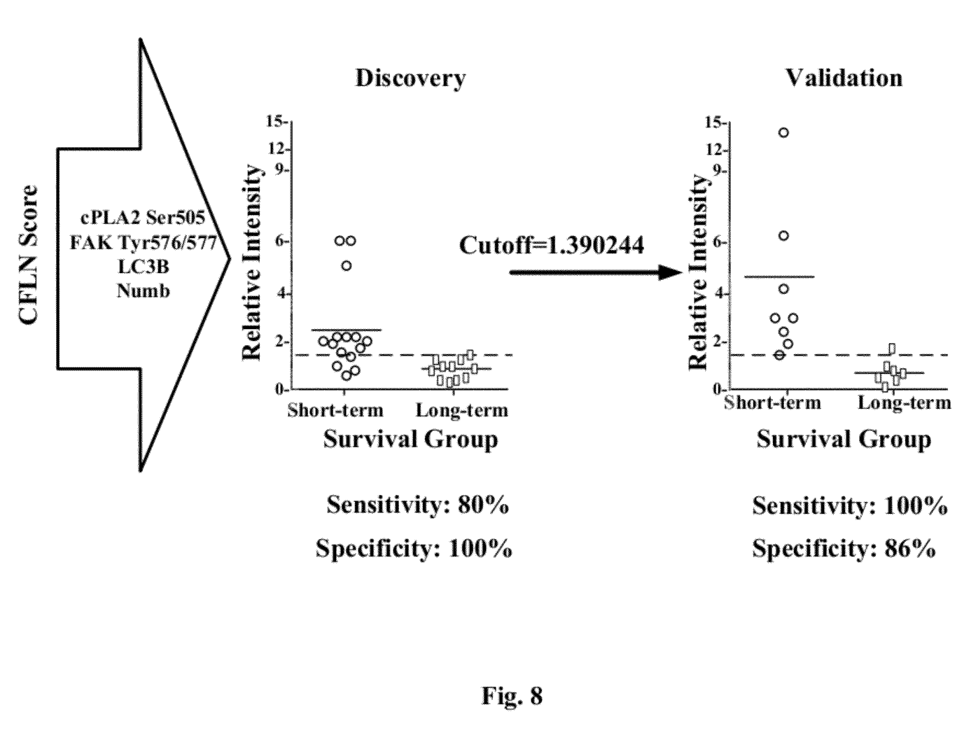

To calculate the rank score values were ranked over the whole population of samples (discovery+validation set). Rank values for proteins that were downregulated in limited survivors (FAK Tyr576/577, Numb) were added together and then subtracted from the sum of the upregulated protein rank values (cPLA2 Ser505, LC3B). (See FIG. 8).

In this specification, “a” and “an” and similar phrases are to be interpreted as “at least one” and “one or more.” References to “an” embodiment in this disclosure are not necessarily to the same embodiment.

Many of the elements described in the disclosed embodiments may be implemented as modules. Additionally, it needs to be emphasized that the above mentioned technologies may be used in combination.

The disclosure of this patent document incorporates material which is subject to copyright protection. The copyright owner has no objection to the facsimile reproduction by anyone of the patent document or the patent disclosure, as it appears in the Patent and Trademark Office patent file or records, for the limited purposes required by law, but otherwise reserves all copyright rights whatsoever.

While various embodiments have been described above, it should be understood that they have been presented by way of example, and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. Thus, the present embodiments should not be limited by any of the above described exemplary embodiments. In particular, it should be noted that, for example purposes, the above explanation has focused on the example(s) drugs suitable for the treatment of Glioblastoma multiforme. However, one skilled in the art will recognize that embodiments of the invention could be other anti-neoplastic agents including Taxol™.

In addition, it should be understood that any figures that highlight any functionality and/or advantages, are presented for example purposes only. The disclosed method is sufficiently flexible and configurable, such that it may be utilized in ways other than that shown. For example, the steps listed in any flowchart may be re-ordered or only optionally used in some embodiments.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

Finally, it is the applicant's intent that only claims that include the express language “means for” or “step for” be interpreted under 35 U.S.C. 112, paragraph 6. Claims that do not expressly include the phrase “means for” or “step for” are not to be interpreted under 35 U.S.C. 112, paragraph 6.

1. A method of selecting a treatment for a patient with a brain neoplasm comprising:
a. incubating a brain tissue sample from said patient with one or more candidate therapeutics;
b. stopping the incubation and applying a fixative to the stopped incubation;
c. determining the activation status of at least one protein; and
d. using the determination to select a treatment that advantageously impacts brain tissue.

2-22. (canceled)
